(19) United States
(12) Patent Application Publication
(10) Pub. No.: US 2026/0196337 A1
(43) Pub. Date: Jul. 9, 2026

(54) SPATIAL RESTRICTIONS FOR AUGMENTED REALITY (AR)/ VIRTUAL REALITY (VR) REMOTE COLLABORATION IN A HOSPITAL ENVIRONMENT

(71) Applicant: KONINKLIJKE PHILIPS N.V., EINDHOVEN (NL)

(72) Inventors: Thomas Erik Amthor, HAMBURG (DE); Christoph Günther Leussler, HAMBURG (DE); Peter Forthmann, SANDESNEBEN (DE)

(21) Appl. No.: 19/129,338

(22) PCT Filed: Nov. 22, 2023
(86) PCT No.: PCT/EP2023/082703

§ 371 Date: May 13, 2025

Related U.S. Application Data
(60) Provisional application No. 63/428,747, filed on Nov. 30, 2022.

(51) Int. Cl. G16H 30/40; G06F 21/62; G06T 5/70; G06T 7/20; G06T 19/00; G06V 20/52; G06V 40/16; G16H 10/60; G16H 40/63; G16H 40/67; G16H 80/00

(52) U.S. Cl. CPC G16H 30/40; G06F 21/6254; G06T 5/70; G06T 7/20; G06T 19/006; G06V 20/52; G06V 40/161; G16H 10/60; G16H 40/63; G16H 40/67; G16H 80/00

(57) ABSTRACT

Monitoring of a medical procedure includes: receiving a video feed of the medical procedure using a mobile camera; receiving camera location tracking data during the recording; determining prohibited image content in the video feed that does not satisfy a predetermined acceptance criterion based on image content analysis and/or the camera location tracking data; editing the video feed to remove or distort the prohibited image content in the video feed; and transmitting the edited video feed to a remote electronic device configured to display the edited video feed. The editing in some cases may include turning off acquisition of the video feed, omitting video frames, removing or blurring the prohibited image content depicting an object or action that does not satisfy the predetermined acceptance criterion, and/or so forth. Editing may also include omitting a portion of the video feed acquired during rapid movement of the mobile camera.

The following relates generally to the imaging arts, remote imaging assistance arts, remote imaging examination monitoring arts, augmented reality arts, virtual reality arts, and related arts.

### BACKGROUND

Medical imaging, such as computed tomography (CT) imaging, magnetic resonance imaging (MRI), positron emission tomography (PET) imaging, fluoroscopy imaging, and so forth, is a critical component of providing medical care, and is used in a wide range of medical fields, such as cardiology, oncology, neurology, orthopedics, to name a few. The operator of the medical imaging device used to acquire the medical images is typically a trained imaging technologist, while interpretation of the medical images is often handled by a medical specialist such as a radiologist.

Currently, medical imaging is in high demand. As the world population ages, the demand for quick, safe, high quality medical imaging will only continue to grow, putting further pressure on imaging centers and their staff. Under such conditions, errors can occur, and can often be costly. One approach for imaging centers to boost efficiency and grow operations at no extra labor costs is through a radiology operations command center (ROCC) system. Radiology operations command centers enable teams to work across the entire network of imaging sites, providing their expertise as needed and remotely assisting less experienced technologists in carrying out high quality scans. Remote technologists or experts can monitor the local operators of scanning procedures through cameras installed in the scanning areas (or from other sources, such as sensors (including radar sensors), console video feeds, microphones connected to Internet of Things (IoT) devices, and so forth. In addition, these sources can be supplemented by other data sources like Health-Level 7 (HL7), Digital Imaging and Communications in Medicine (DICOM), Electronic Health Record (EHR) databases, and so forth.

Moreover, augmented reality/virtual reality (AR/VR) technology can provide remote support for staff working in a hospital environment, for example technologists operating an imaging modality or technical support servicing or exchanging parts. As a specific example, the remote technologist or expert could transmit AR content to an AR headset worn by the local technologist, with simultaneous location and mapping (SLAM) or similar algorithms used to align the AR content with the actual view of the imaging device and/or patient observed by the local technologist.

In order to enable remote support via AR/VR equipment, a video stream recorded by an integrated camera in AR/VR headset or glasses is transmitted to remote support staff, so that the remote staff sees exactly what the local operator sees.

In sensitive environments, such as radiology departments in a hospital, there is a risk that sensitive personal data or other confidential information may be transmitted offsite by the camera. Even if not intended, the local operator could turn their head or even walk into another room with the camera turned on, so that other persons, computer screens, or documents showing sensitive information could be seen by the remote side.

To prevent this from happening, the local operator would need to be aware of where he/she is looking and remove or switch off the AR/VR glasses before turning around to face another person. This is not possible in practice, in particular if the local operator needs both hands for another task at the same time. There is also a risk the local operator may forget to remove or switch off the AR/VR glasses in such situations.

The following discloses certain improvements to overcome these problems and others.

### SUMMARY

In one aspect, a non-transitory computer readable medium stores instructions executable by at least one electronic processor to perform a method of monitoring a medical procedure. The method includes: receiving a video feed of the medical procedure acquired by a mobile camera; receiving camera location tracking data indicating location as a function of time of the mobile camera during the recording of the video feed; determining prohibited image content in the video feed that does not satisfy a predetermined acceptance criterion based on at least one of (i) analysis of image content of the video feed and/or (ii) the camera location tracking data; editing the video feed at least to remove or distort the prohibited image content in the video feed to produce an edited video feed; and transmitting the edited video feed to a remote electronic device configured to display the edited video feed. By way of nonlimiting illustrative examples, the editing may include: turning off acquisition of the video feed by the mobile camera when the video camera is determined to be outside of at least one permitted video recording area; omitting video frames of the video feed recorded when the video camera is determined to be outside of the at least one permitted video recording area from the edited video feed; and/or removing or blurring the prohibited image content depicting an object or action that does not satisfy the predetermined acceptance criterion in video frames of the edited video feed. In some embodiments, the editing further includes detecting a time interval of rapid movement of the mobile camera and omitting the portion of the video feed acquired during the time interval of rapid movement of the mobile camera.

In another aspect, an apparatus is disclosed for monitoring a medical procedure. The apparatus includes a mobile camera configured to acquire a video feed of the medical procedure, a location tracking system configured to acquire camera location tracking data indicating location as a function of time of the mobile camera during the recording of the video feed, and an electronic processor. The electronic processor is programmed to: determine prohibited image content in the video feed that does not satisfy a predetermined acceptance criterion based at least on the camera location tracking data; and edit the video feed at least to remove or distort the prohibited image content in the video feed to produce an edited video feed. In some embodiments the mobile camera includes a head-mounted camera worn by a medical professional performing the medical procedure. In some embodiments the location tracking system includes a sensor configured to acquire camera direction tracking data as a function of time, and the electronic processor is programmed to determine the prohibited image content in the video feed based on the camera location tracking data including the camera direction tracking data.

In another aspect, a method of monitoring a medical procedure performed on a patient is disclosed. The method includes: receiving a video feed of the medical procedure acquired by a mobile camera; receiving camera location tracking data indicating location as a function of time of the mobile camera during the recording of the video feed; determining patient-identifying information in the video feed based at least on analysis of image content of the video feed; editing the video feed at least to remove or distort the patient-identifying information in the video feed to produce an edited video feed; and transmitting the edited video feed to a remote electronic device configured to display the edited video feed.

One advantage resides in providing a remote expert with a field of view of a local operator performing a medical examination via an augmented reality device.

Another advantage resides in preventing sensitive information from being viewed during a medical examination.

Another advantage resides in automatically turning off a camera (or transmission of the video feed) of an augmented reality device used during a medical procedure at appropriate times during the medical procedure to prevent sensitive information from being transmitted offsite or viewed by an unauthorized person.

Another advantage resides in determining sensitive information or objects that should not be viewed by someone not performing a medical examination.

The ability to perform certain useful real-time control of the medical imaging device while preventing unintentional medical device interactions by the remote user during a remote imaging assistance session.

Another advantage resides in preventing a remote user from viewing sensitive data during a remote imaging assistance session.

Another advantage resides in preventing hacking of data during a remote imaging assistance session.

Another advantage resides in restricting a USB remote operation of a device to certain allowed actions by means of a hardware solution that is only controllable by the local side, thereby preventing a remote user from (unintended) accessing of certain information or functionality.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows another embodiment of the apparatus of FIG. 1.

FIGS. 3 and 4 show example flow charts of operations suitably performed by the apparatus of FIG. 1.

### DETAILED DESCRIPTION

As part of ongoing Remote Operations Control Center (ROCC) development, the use of augmented reality (AR) or virtual reality (VR) is contemplated as a way to better integrate the remote expert into the workflow. An AR or VR headset typically includes an outward facing camera that captures the view of the wearer. The local imaging technician can thus wear the AR or VR headset and the remote expert can see exactly what the local imaging technician is seeing via the headset-mounted camera. The AR or VR capability of the headset can be used to provide the local imaging technologist with graphical overlays or the like supplied by the remote expert to guide the imaging technologist in performing the imaging examination.

However, a risk with such a setup is that the camera may capture content that should not be transmitted offsite, such as images of the patient, medical images of the patient, or so forth potentially compromising patient privacy, or images of the hospital environment that may be subject to trade secret or other restriction. Other perhaps less serious but still problematic issues could arise, such as if the camera continues to broadcast while the local imaging technologist takes a bathroom break.

To address these concerns, the following discloses a system to restrict the camera video content that is broadcast to the remote expert (or other recipient). Initially, a permitted location of the hospital or other medical facility is identified, such as the imaging bay where the medical imaging examination is being performed (possibly including an adjacent control room). A map of the hospital identifies the permitted location (or possibly permitted locations), and everywhere else is deemed restricted. The AR or VR headset includes or is in communication with a real-time locating service (RTLS) or other location tracking system that tracks its location, and the camera (or at least the camera feed to the remote expert) is automatically turned off if the headset leaves the imaging bay or other permitted location.

Even in the permitted location (e.g. the imaging bay) there may be areas that should be restricted. For example, the patient's face, certain display screens containing patient-identifying information (PII, e.g., patient name, patient age, hospital patient identifier, a medical file with the patient's name and/or medical history, an identification (ID) card containing information about the patient, or so forth) or trade secret information, or the like should not be broadcast to the remote expert. In one approach to handle this, the headset includes a head direction tracker (e.g., a gyroscope or the like) to track the viewing direction of the camera. Combined with the location information and the map, this information can identify if the wearer is looking in a prohibited direction, such as at a prohibited display or into the scanner bore where the patient's head is located, and if so the camera (or, again, at least the camera feed to the remote expert) is turned off.

This directional-based approach can have limitations, however. For example, it cannot handle the case of overlapping objects in which one object is a permitted camera viewing target, but the other object is a prohibited camera viewing target. To handle such situations, the system additionally or alternatively includes an image analysis-based restriction approach. In this approach frames of the camera video are analyzed to perform object identification using region-based convolutional network (R-CNN) or fully convolutional neural networks (R-FCN), you-only-look-once (YOLO) algorithm, or so forth. The camera frame can thus be divided into permitted and prohibited image regions. The restriction can be implemented by turning off the camera (or the video feed) in response to any image region capturing a prohibited area. Alternatively, the restriction can be implemented by performing image processing on the image frame to replace the prohibited image region(s) with a suitable filler image or pattern or the like, or the prohibited image region can be blurred or otherwise obfuscated. To provide time for the image analysis, it may be appropriate to include a short delay Δt in the camera feed transmitted to the remote expert, and/or to analyze a subset of the camera video frames to reduce processing load. For example, if Δt=1 second and the camera is operating at 60 frames/second (fps) and every tenth frame is analyzed, then this provides a window of ⅚th of a second (833 milliseconds) to perform the image processing and insert regional obfuscation as needed. The same obfuscation can be inserted into the unanalyzed frames captured after the analyzed frame up to the transmission time (due to the offset Δt) under the expectation that the camera movement should be relatively slow. The obfuscation can also be slightly extended (e.g., extending 5% larger than the border of the restricted image region) to accommodate small frame-to-frame changes between the analysis frames.

To accommodate rapid head movements, a time interval of rapid movement of the head-mounted camera can be detected, and the portion of the video feed acquired during the time interval of rapid movement of the head-mounted camera can be omitted from the transmitted video feed. In one approach for detecting a time interval of rapid movement of the head-mounted (or other mobile) camera, content of successive frames can be compared using a fast comparison such as summing the change in corresponding pixel intensities over all pixels. If the content change is larger than some threshold (indicating the head is rapidly turning) then the transmitted video feed can be turned off until the content stabilizes in time. This prevents single frames that inadvertently capture something restricted during a head turn from being transmitted in the video feed.

A range finder or three-dimensional (3D) camera could also be used to separate (or help to separate) overlapping objects by distance, and similar obfuscation can be applied to image content at a distance corresponding to a prohibited object. In a variant (and not necessarily mutually exclusive) approach, content at a distance greater than a pre-selected threshold distance can be automatically blurred or otherwise obfuscated, since distant content is more likely to inadvertently contain something prohibited and is unlikely to contribute meaningfully to the situational understanding of the remote expert.

The disclosed approaches can be applied to any head-mounted camera (with or without ROCC), and even more broadly to any mobile camera (e.g., mounted on a medical instrument). The disclosed approaches are applicable to medical imaging as well as to other medical treatment settings such as remote surgery, telehealth, and the like that can benefit from the use of a camera providing a feed to a remote expert (e.g., the remote surgeon or remote doctor in these examples). Additionally, the disclosed approaches are applicable to field servicing of medical devices. For example, a field service engineer (FSE) visiting a hospital to perform maintenance on a medical imaging device (or other medical device) could use a camera to provide a feed to a remote service technician providing assistance to the FSE. In this case, the permitted location would suitably be the location of the medical imaging device under service, and the permitted areas in that location would typically include the imaging device under service. In some cases, some parts of the imaging device might be prohibited (for example, if they constitute trade secret technology), in which case the image analysis based obfuscation can be applied for those areas.

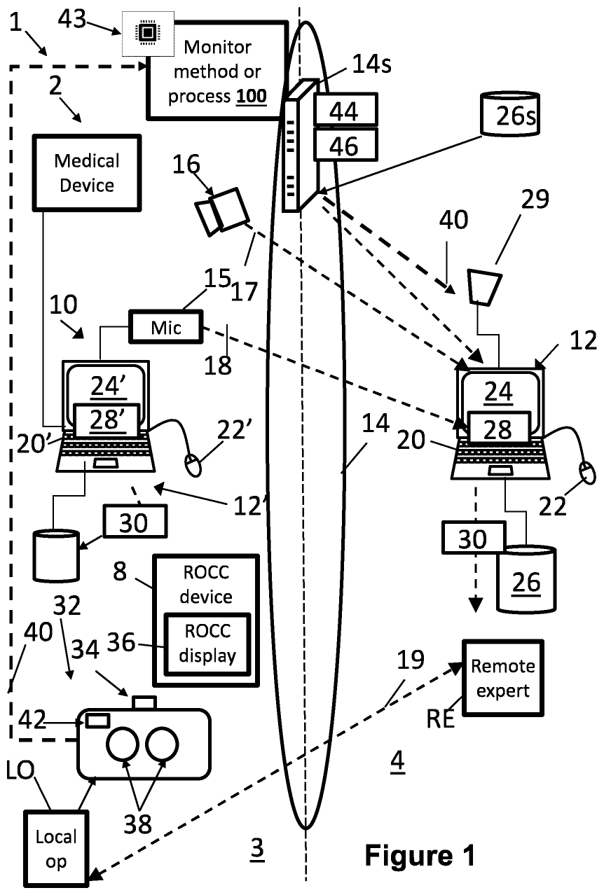

With reference to FIG. 1, an apparatus 1 for providing assistance from a remote medical imaging expert RE (or supertech) to a local technologist operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator or expert” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. Moreover, the remote service center 4 may provide service to multiple hospitals. The local operator controls the medical imaging device 2 via an electronic imaging device controller 10. The remote operator is stationed at an assistance electronic device 12 (or, more generally, a remote workstation 12 or an electronic controller 12).

Inputs from the electronic imaging device controller 10 can be sent to the remote electronic controller 12 via a communication link 14, e.g., as a streaming video feed received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14, e.g., the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via a local electronic processing device, for example comprising an ROCC device 8, such as a mobile device (e.g., a tablet computer or a smartphone), or can be a wearable device worn by the local operator LO, such as an augmented reality (AR) display device (e.g., AR goggles), a projector device, a heads-up display (HUD) device, etc., each of which having a display device 36. For example, an “app” can run on the ROCC device 8 (operable by the local operator LO) and the assistance electronic device 12 (operable by the remote expert RE) to allow communication (e.g., audio chats, video chats, and so forth) between the local operator and the remote expert.

FIG. 1 also shows, in the remote service center 4 including the assistance electronic device 12, such as an electronic processing device, a workstation computer, or more generally a computer, which is operatively connected to receive and present the video feed of the medical imaging device bay 3 from the camera 16 and/or to the audio feed from the microphone 15. Additionally or alternatively, the remote electronic processing device 12 can be embodied as a server computer or a plurality of server computers, e.g., interconnected to form a server cluster, cloud computing resource, or so forth. The remote electronic processing device 12 includes typical components, such as an electronic processor 20 (e.g., a microprocessor), at least one user input device 22, such as an illustrative mouse, a keyboard, a trackball, trackpad, touch-sensitive display, or so forth. The remote electronic processing device 12 further includes at least one display device 24 (e.g., an LCD display, plasma display, and/or so forth). In some embodiments, the display device 24 can be a separate component from the remote electronic processing device 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the remote electronic processing device 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed from the camera 16 can also be displayed on the display device 24, and the audio feed from the microphone 15 can be output on the assistance electronic device 12 via a loudspeaker 29. In some examples, the audio feed can be an audio component of an audio/video feed (such as, for example, recording as a video cassette recorder (VCR) device would operate).

FIG. 1 shows an illustrative local operator LO, and an illustrative remote expert RE (e.g., supertech). However, in a Radiology Operations Command Center (ROCC) as contemplated herein, the ROCC provides a staff of supertechs who are available to assist local operators LO at different hospitals, radiology labs, or the like. Each remote expert RE can operate a corresponding remote electronic processing device 12. The ROCC may be housed in a single physical location or may be geographically distributed. For example, in one contemplated implementation, the remote expert RE are recruited from across the United States and/or internationally in order to provide a staff of supertechs with a wide range of expertise in various imaging modalities and in various imaging procedures targeting various imaged anatomies. A server computer 14s can be in communication with the medical imaging bay 3 and the remote service center 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The medical imaging device controller 10 in the medical imaging device bay 3 also includes similar components as the remote electronic device 12 disposed in the remote service center 4. Except as otherwise indicated herein, features of the medical imaging device controller 10, which includes a local workstation 12′, disposed in the medical imaging device bay 3 similar to those of the assistance electronic device 12 disposed in the remote service center 4 have a common reference number followed by a “prime” symbol, and the description of the components of the medical imaging device controller 10 will not be repeated. In particular, the medical imaging device controller 10 typically includes a keyboard 21′ and at least one pointing device 22′, such as an illustrative mouse 22′, or a trackball, trackpad, touch-sensitive display, or so forth. The medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the controller display screen mirroring data stream (i.e., controller display video) 17 carries the content presented on the display device 24′ of the medical imaging device controller 10. The communication link 14 allows for screen sharing of the controller display video 17 from the controller 10 to the remote workstation 12. The GUI 28′ running on the medical imaging device controller 10 includes one or more dialog screens, including, for example, an examination/scan selection dialog screen, a scan settings dialog screen, an acquisition monitoring dialog screen, among others. The GUI 28′ can be included in the video feed 17 and displayed on the assistor electronic device display 24 of the remote workstation (i.e., assistor electronic device) 12 at the remote location 4.

FIG. 1 also shows a device 32 operable by the local operator LO to provide the remote expert RE with a “first-person” view of the medical imaging device bay 3. The device 32 includes a stereo camera 34 configured to acquire (optionally stereo) images or videos of the medical device 2 and/or locations of structures or objects within the medical imaging device bay 3. The illustrative stereo camera 14 typically includes multiple lenses or lens assemblies with a separate sensor for each lens that forms an image on a digital detector array (e.g., a CCD imaging array, a CMOS imaging array, et cetera) to capture 3D images. The stereo camera 14 preferably (although not necessarily) has color video capability, e.g., by having an imaging array with pixels sensitive to red, green, and blue light (or another set of colors substantially spanning the visible spectrum, e.g., 400-700 nm). The stereo camera 14 optionally may include other typical features, such as depth detection, a built-in flash (not shown) and/or an ambient light sensor (not shown) for setting aperture, ISO, and/or exposure times. While a stereo camera is described, a single-lens non-stereo camera could also be used as the camera 34.

In some embodiments, the device 32 comprises an augmented reality-heads up display (AR-HUD) device 32 wearable by the local operator LO. The AR-HUD device 32 includes one or more AR-HUD displays 38 comprising lenses of the AR-HUD device 32. The illustrative design employs left-eye and right-eye displays 38, but alternatively the display can be a single large window that spans both eyes. In some examples, the stereo camera 34 is mounted to the AR-HUD device 32 to provide a “first person view” video feed 40 that is transmitted from the AR-HUD device 32 to the server computer 14s for processing, and then to the remote electronic processing device 12 to allow the remote expert RE to “see” the field of view of the local operator LO wearing the AR-HUD device 32. In some examples, the AR-HUD device 32 can be configured as a helmet, a headband, glasses, goggles, or other suitable embodiment in order to be worn on the head of the user. The stereo camera 34 is mounted to the AR-HUD device 32 (e.g., to overlay the user's forehead, or including two stereo cameras disposed on lenses of the glasses). The AR-HUD device 32 also includes one or more sensors 42 configured to acquire position data of, for example, the local operator LO, objects in the medical imaging device bay 3, and so forth. In one example, the sensor(s) 42 can comprise a gyroscope 42 configured to obtain field of view data of the AR-HUD device 32. In another example, the sensor(s) 42 can comprise a real-time locating service (RTLS) sensor 42 configured to acquire location information of the AR-HUD device 32. The acquired location information can be compared with a map 44 of the medical imaging device bay 3 stored in the server computer 14s.

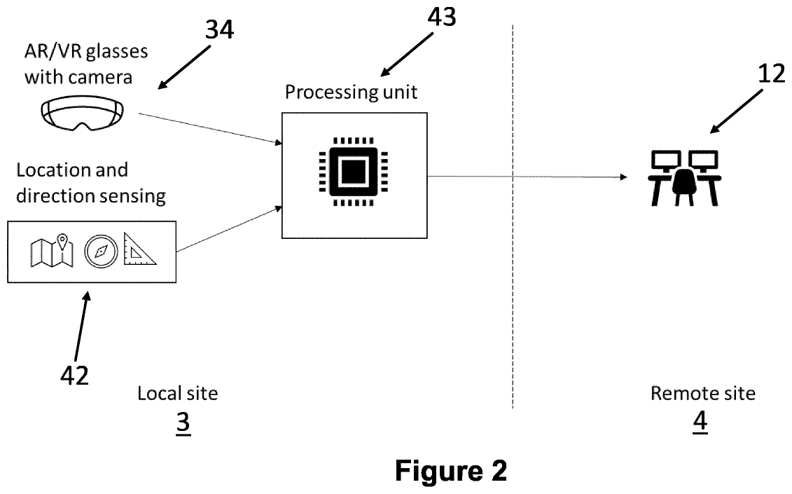

With continuing reference to FIG. 1 and with further reference to FIG. 2, furthermore, as disclosed herein, an electronic processor 43 performs a method or process 100 for monitoring a medical procedure performed using a medical imaging device 2 (i.e., by assisting local operators LO of respective medical imaging devices 2 during medical imaging examinations by a remote expert RE). The instructions to perform the method 100 are stored in a non-transitory computer readable medium (not shown). The method 100 includes editing a video feed recorded at the local site 3 by the camera 34 (e.g., embodied as AR/VR glasses with camera in FIG. 2) to redact sensitive image content and then transmitting the edited video feed offsite, e.g., to a remote site (for example, to the assistance electronic device 12 disposed in the remote service center 4). To ensure the sensitive image content is redacted before transmission offsite, in some embodiments the electronic processor 43 is located at the local site 3. For example, the electronic processor 43 may be integrated with the AR/VR glasses, or may be implemented as a processor of the ROCC device 8, or so forth. Alternatively, the electronic processor 43 could be located offsite—in this case the video feed is preferably strongly encrypted before transmission offsite where the encrypted video feed is decrypted using a public/private key pair or other suitable encryption/decryption technology. If located offsite, the electronic processor 43 should be highly secure. In an example of an offsite arrangement, the electronic processor 43 could be embodied as the server computer 14s which preferably has a strong firewall and other suitable security provisions.

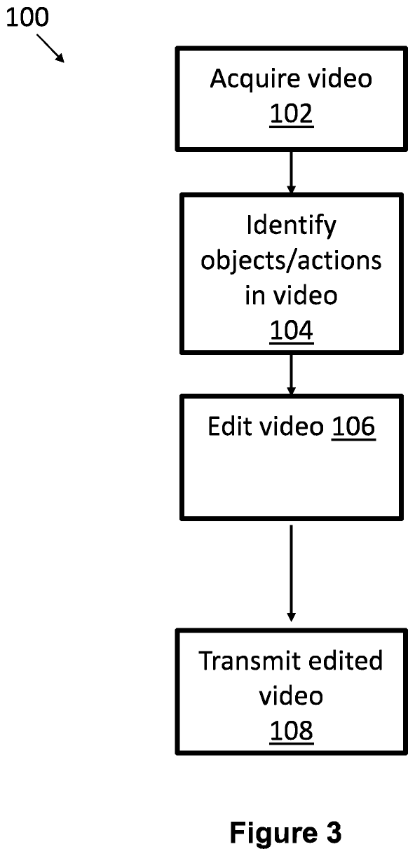

With reference now to FIG. 3 which shows an embodiment of the method 100, at an operation 102, a video feed of content of objects in the medical device imaging bay 3, or actions performed in the medical device imaging bay 3 by, for example, the local operator LO. In one example, the video feed 17 is acquired by the camera 16. In a particular example, the video feed 40 is acquired by the AR-HUD device 32 worn by the local operator LO. In some embodiments, field of view information of the stereo camera 34 can be obtained from the gyroscope 42. In other (non-mutually exclusive) embodiment, location information of the stereo camera 34 can be obtained from RTLS sensor 42 and compared with the map 44. In another example embodiment, the video feed 40 can include overlapping objects. These overlapping objects can be separated, for example, by using imaging data from a three-dimensional (3D) camera (i.e., the camera 16). The video feed 40 is then transmitted to the server computer 14s for processing.

At an operation 104, objects or actions in the video feed 40 that do not satisfy a predetermined acceptance criterion are determined, and at an operation 106, the video feed 40 can be edited to remove or distort the determined objects or actions in the video feed that do not satisfy the predetermined acceptance criterion. These operations 104, 106 can be performed in a variety of manners. In one example embodiment, the building where the medical procedure is taking place (i.e., the medical imaging device bay 3) can be determined, for example by using data acquired the RTLS sensor 42 and analyzed with the map 44. Locations within the building that do not satisfy the predetermined acceptance criterion can be identified (i.e., an emergency exit stairwell, a medical device bay where the imaging device 2 is not located, and so forth). When the AR-HUD device 32 is determined to be in a location that does not satisfy the predetermined acceptance criterion, the camera 34 can be controlled by the server computer 14s and turned off. The location of the AR-HUD device 32 can be continuously determined, and the camera 34 can be turned back on when the AR-HUD 32 is determined to be in a permitted location. In another example, the camera 34 can record actions performed by the local operator LO (i.e., steps in the medical examination). In another example, the objects or actions in the video feed 40 that do not satisfy a predetermined acceptance criterion can include patient-identifying information (PII) about a patient. For example, prohibited information comprising PII can be identified as a face depicted in the video feed 40 (in some embodiments, any face is blurred or removed; in other embodiments, only the face of the patient identified based on his or her clothing, location, or so forth is blurred or removed). In another example, prohibited information comprising PII such as the patient name is identified in video depicting the display device 24′ of the medical imaging device controller 10 based on location and/or an associated label such as “Patient:”. The camera 34 can be controlled by the server computer 14s and turned off responsive to any action performed during the examination (i.e., the patient undressing) that does not satisfy the predetermined acceptance criterion.

In another example embodiment, a field of view of the AR-HUD device 32 can be determined, for example, by using data acquired by the gyroscope 42. One or more objects in the field of view of the camera 34 that do not satisfy the predetermined acceptance criterion (i.e., a file containing personal information about a patient undergoing a medical procedure, a face of the patient, and so forth). When such objects are identified, the camera 34 can be controlled by the server computer 14s and turned off. The location of the AR-HUD device 32 can be continuously determined, and the camera 34 can be turned back on when the AR-HUD 32 is determined to be not showing such objects.

The operation 104 can comprise analyzing the video feed 40 with an object identification process or action identification process to identify objects/actions in the video feed 40 by analyzing individual image frames in the video feed 40. This analysis can be performed by an artificial intelligence (AI) component 46 (e.g., an artificial neural network (ANN)) stored and executed by the server computer 14s. The individual video frames can each be classified by the AI component 46 as (i) objects or actions that do not satisfy the predetermined acceptance criterion and (ii) objects or actions that satisfy the predetermined acceptance criterion.

The editing operation 106 can then be performed in a variety of manners. In one example, the video feed 40 can be edited to include only the (ii) objects or actions that satisfy the predetermined acceptance criterion. In another example, the camera 34 can be turned off in response to frames including (i) objects or actions that do not satisfy the predetermined acceptance criterion. To do so, for example, content of the image frames can be compared to each other by summing a change in pixel intensity in each pixel over all of the frames. The camera 34 can then be turned off when a sum of the change in pixel intensity is larger than a predetermined intensity threshold.

At an operation 108, the edited video feed 40 is transmitted to the remote electronic processing device 12 for viewing by the remote expert RE. In some examples, the edited video feed 40 can include a time delay in transmitting the edited video feed 40 to the remote electronic processing device 12 to ensure that all “unpermitted” objects or actions are removed from the edited video feed 40. The remote expert RE can then review the edited video feed 40, and if the local operator LO requires assistance from the remote expert RE, then the natural communication pathway 19 can be established between the local operator LO and the remote expert RE.

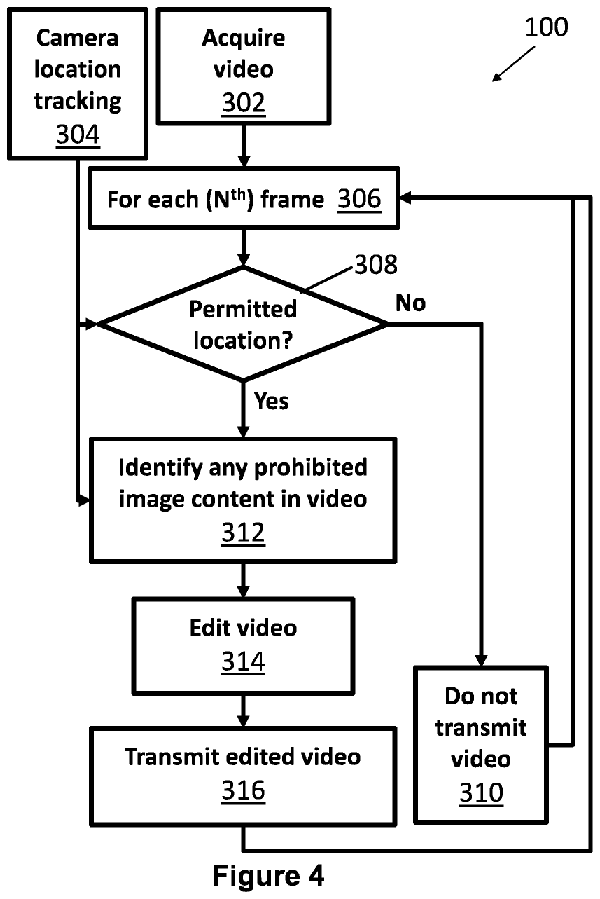

With reference to FIG. 4, another embodiment of the method 100 is shown. In this embodiment the video feed 302 is acquired using the camera 34, along with camera location tracking 304 employing an RTLS or other tracking system that acquires camera location tracking data indicating location as a function of time of the mobile camera 34 during the recording of the video feed. Each video frame 306 (or, to simplify processing, for each Nth video frame, e.g., for every fifth video frame in one example providing ⅙th second resolution at 30 fps video) is processed. In an operation 308 it is determined whether the video frame is acquired at a permitted location. To this end a map indicating at least one permitted video recording area is provided (e.g., stored in a non-transitory storage medium), and the determining of prohibited image content in the video feed includes determining from the camera location tracking data 304 when the video camera 34 is outside of the at least one permitted video recording area. If the camera is not in a permitted location at the time the video frame was acquired, then in an operation 310 that video frame is not transmitted. (If every Nth video frame is being processed, then to be safe a group of N−1 following frames may also be not transmitted as well).

If the decision 308 determines the camera 34 is recording the video frame 306 at a permitted location, then in an operation 312 any prohibited image content in the video is identified. This can be done by image analysis, for example by performing object detection using an R-CNN, R-FCN, YOLO, or the like. If the video frame 306 is determined at operation 312 to contain prohibited image content, then in an operation 314 the video is edited. For example, the video frame can simply be omitted from the edited video. Alternatively, the prohibited image content comprising a prohibited object or action detected by the object detection can be removed (e.g., replaced by a background pattern) or blurred in the image frame 306. Again, if only every Nth video frame is analyzed then such remediation is suitably performed on the next N−1 frames which are not analyzed. (In this case, the area that is removed or blurred can be enlarged by a suitable extension, e.g., a 5% area enlargement, to accommodate small changes in camera position over the N−1 succeeding frames).

Optionally, the editing 314 can include other types of editing to ensure sensitive content is not transmitted. For example, the editing 314 can include blurring distant image content (for example, as determined using a 3D camera 34) as distant image content is unlikely to be what the local technician is focusing on, and such blurring greatly reduces the likelihood of inadvertently transmitting something sensitive occupying the background. As another example, the editing 314 may include detecting whether the video frame 306 is part of a time interval of rapid movement of the mobile camera, and if so then the video frame 306 can be omitted from the edited video. Again, this provides protection against inadvertently transmitting something sensitive occupying a large field-of-view swing caused by the rapid camera movement. As previously noted, if only every Nth video frame is analyzed then such remedial actions can be extended over the N−1 succeeding frames.

Finally, in an operation 316, the edited video feed is transmitted to the remote site. Optionally, this can be done with a time delay Δt chosen to ensure there is sufficient time to perform the operations 308, 312, and 314 before the edited video frame is transmitted.

In the following, some further examples are provided.

### Examples

The following describes the ROCC apparatus 1 in more detail. At the medical imaging device bay 3 (e.g., a radiology department), the local operator LO is wearing the AR-HUD device 32. Location and direction sensors 42 attached to the AR-HUD device 32 track the position and viewing direction of the camera 34. The server computer 14s modifies the video stream 40 according to the location and direction information. The resulting modified video stream 40 is then transmitted to the remote expert RE.

Location and direction sensing can partly be achieved by the one or more sensors of the AR-HUD device 32, such as gyroscopes, compasses, or accelerometers. In some environments, such as MR modalities, compass sensors will not be able to work. Direction sensing can be achieved with the sensor(s) 42 being attached to the AR-HUD device 32 itself.

General location tracking (to identify which room the local operator is in) can be achieved using a Real-Time Location Sensing (RTLS) sensor 42, or pilot tone emitters/receivers attached to the AR-HUD device 32).

Further location detection of the user can be done by Ultra-Wide Band (UWB) localization, such as low power pilot tones for position detection in the clinical environment. A microwatt transmitter is integrated in the AR-HUD device 32, so that a person can be clearly detected and identified in a clinical environment, thus further data privacy measures can be applied for the local operator LO and also for the remote expert RE.

The definition of which areas in a location are allowed to be viewed can be realized using a 2D (floor plan) or 3D (volume) CAD application such as the map 44, where a used marks areas allowed to be viewed. In one embodiment, several regions can be defined for different purposes, so that the applicable regions can be selected from the current application context. Alternatively, instead of defining allowed regions, a user could also define restricted areas, which would then identify all other areas as allowed. The geometry of allowed and restricted areas is stored in the electronic map 44 of the medical imaging device bay 3.

Alternatively, the AR-HUD device 32 only start working after the local operator LO has set the view direction at the beginning of the medical procedure. This plus a predefined moving radius then sets the allowed area. If the local operator LO, then turns their head beyond a certain limit or move out of some predefined radius, the AR-HUD device 32 stop transmitting the camera feed. The local operator LO could also receive a warning that they are out of range. In the simplest case, the warning can be an efficient reminder to turn the AR-HUD device 32 off manually.

The server computer 14s collects location and direction information and maps this information to the electronic site map 44 that includes allowed and restricted areas. The server computer 14s further calculates which part of the camera image will thus view an allowed or restricted area.

The video stream 40 can then be processed in different ways, including, but not limited to, complete blocking of the video stream 40 as soon as a restricted area is in view, complete blocking of the video stream 40 if a restricted area is in view and closer than a pre-defined distance (would allow the video to be transmitted when looking at an imaging modality while a few meters further in the background there is a restricted area, which would be out of focus anyway), cutting out restricted parts of the image and replacing them with an overlay (e.g., black box), blurring restricted parts of the image, cutting out or blurring of restricted areas if they are closer than a pre-defined distance, and so forth.

When restricted areas are in view and the video stream 40 is cut, blurred, or blocked, a message or symbol can be viewed on the remote electronic processing device 12 to indicate that viewing restrictions apply to the current image.

In one embodiment, in addition to the pre-defined spatial restricted areas, the AI component 46 implemented in the server computer 14s may detect faces or other sensitive information in any part of the image, so that these areas can also be cut out or blurred automatically for additional safety.

Under some circumstances, some parts of the technical setup may be considered restricted. For example, when a local technician has opened a cover of a device for servicing and there is a risk of revealing technical secrets. In this case, additional restricted areas or AI detection methods can be added.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a method of monitoring a medical procedure, the method comprising:
receiving a video feed of the medical procedure acquired by a mobile camera;
receiving camera location tracking data indicating location as a function of time of the mobile camera during the recording of the video feed;
determining prohibited image content in the video feed that does not satisfy a predetermined acceptance criterion based on at least one of (i) analysis of image content of the video feed and/or (ii) the camera location tracking data;
editing the video feed at least to remove or distort the prohibited image content in the video feed to produce an edited video feed; and
transmitting the edited video feed to a remote electronic device configured to display the edited video feed.

2. The non-transitory computer readable medium of claim 1, further storing:
a map indicating at least one permitted video recording area;
wherein the determining of prohibited image content in the video feed includes determining from the camera location tracking data when the video camera is outside of the at least one permitted video recording area.

3. The non-transitory computer readable medium of claim 2 wherein the editing of the video feed includes turning off acquisition of the video feed by the mobile camera when the video camera is determined to be outside of the at least one permitted video recording area.

4. The non-transitory computer readable medium of claim 2, wherein the editing of the video feed includes omitting video frames of the video feed recorded when the video camera is determined to be outside of the at least one permitted video recording area from the edited video feed.

5. The non-transitory computer readable medium of claim 2, wherein the camera location tracking data comprises real-time locating service (RTLS) data.

6. The non-transitory computer readable medium of claim 1, wherein the method further includes:
receiving camera direction tracking data as a function of time from a sensor operatively connected to the mobile camera;
determining a field of view of the mobile camera based on the camera direction information and the camera location tracking data;
determining the prohibited image content based on one or more objects being in the determined field of view of the mobile camera that do not satisfy the predetermined acceptance criterion.

7. The non-transitory computer readable medium of claim 1, wherein determining prohibited image content in the video feed that does not satisfy a predetermined acceptance criterion includes:
analyzing image content of the video feed to identify prohibited image content depicting an object or action that does not satisfy the predetermined acceptance criterion.

8. The non-transitory computer readable medium of claim 7, wherein editing the video feed includes:
omitting video frames of the video feed containing the prohibited image content depicting the object or action that does not satisfy the predetermined acceptance criterion from the edited video feed.

9. The non-transitory computer readable medium of claim 7, wherein editing the video feed includes:
removing or blurring the prohibited image content depicting the object or action that does not satisfy the predetermined acceptance criterion in video frames of the edited video feed.

10. The non-transitory computer readable medium of claim 7, wherein the analyzing is performed by an artificial intelligence (AI) component.

11. The non-transitory computer readable medium of claim 1, wherein determining prohibited image content in the video feed that does not satisfy a predetermined acceptance criterion includes:
analyzing image content of the video feed to identify prohibited image content depicting patient-identifying information about a patient undergoing the medical procedure.

12. The non-transitory computer readable medium of claim 1, wherein the editing of the video feed to produce the edited video feed further includes:
detecting a time interval of rapid movement of the mobile camera; and
omitting the portion of the video feed acquired during the time interval of rapid movement of the mobile camera.

13. The non-transitory computer readable medium of claim 12, wherein the detecting of the time interval of rapid movement of the camera includes:
comparing content of successive video frames of the video feed by summing a change in corresponding pixel intensity between the successive video frames over all pixels of the video frames; and
detecting the time interval of rapid movement of the camera based on the comparisons.

14. An apparatus for monitoring a medical procedure, the apparatus comprising:
a mobile camera configured to acquire a video feed of the medical procedure;
a location tracking system configured to acquire camera location tracking data indicating location as a function of time of the mobile camera during the recording of the video feed; and
an electronic processor programmed to:
determine prohibited image content in the video feed that does not satisfy a predetermined acceptance criterion based at least on the camera location tracking data; and
edit the video feed at least to remove or distort the prohibited image content in the video feed to produce an edited video feed.

15. The apparatus of claim 14, wherein:
the mobile camera comprises a head-mounted camera worn by a medical professional performing the medical procedure.

16. The apparatus of claim 14, wherein:
the location tracking system includes a sensor configured to acquire camera direction tracking data as a function of time; and
the electronic processor is programmed to determine the prohibited image content in the video feed based on the camera location tracking data including the camera direction tracking data.

17. A method of monitoring a medical procedure performed on a patient, the method comprising:
receiving a video feed of the medical procedure acquired by a mobile camera;
receiving camera location tracking data indicating location as a function of time of the mobile camera during the recording of the video feed;
determining patient-identifying information in the video feed based at least on analysis of image content of the video feed;
editing the video feed at least to remove or distort the patient-identifying information in the video feed to produce an edited video feed; and
transmitting the edited video feed to a remote electronic device configured to display the edited video feed.

18. The method of claim 17, wherein determining patient-identifying information in the video feed includes:
analyzing image content of the video feed to identify a face in the video.

19. The method of claim 17, wherein determining patient-identifying information in the video feed includes:
analyzing image content of the video feed to identify a name of the patient depicted in the video.

20. The method of claim 17, wherein the medical professional performing the medical procedure is a local operator (LO) of a medical device, the remote electronic processing device is operable by a remote expert (RE), and the method further includes:
establishing a natural communication pathway between the local operator (LO) and a remote expert (RE) based on the transmitted video feed.
